(19) United States
(12) Patent Application Publication
(10) Pub. No.: US 2026/0306968 A1
(43) Pub. Date: Oct. 8, 2026

(54) METHOD FOR THE SEPARATION OF PHOSPHORUS STEREOISOMERS OF REMDESIVIR USING LIQUID-LIQUID CHROMATOGRAPHY

(71) Applicant: ROTACHROM TECHNOLÓGIAI ZRT., Kecskemét (HU)

(72) Inventors: Gergo DARGÓ, Gyál (HU); Dóra RUTTERSCHMID, Dabas (HU); Árpád KÖNCZÖL, Maglód (HU); Zsolt KOVÁCS, Újhartyán (HU); László Tamás ÓVÁRI, Pákozd (HU)

(73) Assignee: ROTACHROM TECHNOLÓGIAI ZRT., Kecskemét (HU)

(21) Appl. No.: 19/482,379

(22) PCT Filed: Jan. 31, 2024
(86) PCT No.: PCT/HU2024/050006

§ 371 Date: Nov. 7, 2025

(30) Foreign Application Priority Data
May 9, 2023 (HU) P2300153

(51) Int. Cl. B01D 15/42; B01D 15/18; C07H 1/06

(52) U.S. Cl. CPC B01D 15/426; B01D 15/1894; C07H 1/06

(57) ABSTRACT

The invention relates to a method for the separation of R_{P} and/or S_{P} remdesivir from a R_{P}-S_{P} isomeric mixture by liquid-liquid chromatography (LLC) using a two-phase, binary, ternary or quaternary solvent system.

Binary solvent systems consist of an alkanoate ester with 3-6 carbon atoms and an aqueous solution containing a water solubility-enhancing additive and a pH-regulating excipient. The ternary solvent systems consist of an alkane with 5-8 carbon atoms, a furane-type aromatic heterocyclic compound or an alkanoate ester with 3-6 carbon atoms, and an aqueous solution containing a water-solubility-enhancing additive and/or a pH-value regulating excipient. The quaternary solvent systems consist of an alkane with 5-8 carbon atoms, a furane-type aromatic heterocyclic compound and an additional third organic co-solvent component. The ternary solvent systems may contain an aqueous solution containing a water-solubility-enhancing additive and/or a pH-regulating excipient, or may consist of pure water alone without any additives.

### FIELD OF THE INVENTION

This invention relates to a novel separation process, in particular to a process for the separation of Rₚ and S_{P} phosphorus stereoisomers of remdesivir using liquid-liquid chromatography, allowing for the straightforward production of the S_{P} isomer and the uncommon R_{P} isomer from their isomeric mixture.

### BACKGROUND OF THE INVENTION

Remdesivir is a nucleoside monophosphoramidate prodrug (ProTide) from the family of viral RNA-polymerase inhibitors, which was developed by Gilead Sciences (GS-5736), initially for the treatment of diseases caused by the Ebola virus, during which it reached phase III clinical trials. Later, it was also applied as an experimental treatment in the fight against other single-stranded RNA viruses, where it played an outstanding role in the fight against the SARS-COV-2 virus, which caused the Covid-19 disease.

The antiviral drug Veklury, which contains the S_{P} isomer of remdesivir as an active pharmaceutical ingredient (API), has been granted the emergency use authorization (EUA) for the investigational antiviral remdesivir to treat COVID-19 by the U.S. Food and Drug Administration (FDA) (May 2020), which was later extended to full approval (October 2020). This was followed by the conditional approval (July 2020) of the European Medicines Agency based on the recommendation of the Committee for Medicinal Products for Human Use (CHMP), which was later switched to full approval (August 2022).

In cells, the remdesivir prodrug is transformed into the pharmacologically active nucleoside triphosphate metabolite, which exerts its effect by inhibiting the synthesis of viral RNA, thus preventing the growth of various viruses, e.g., the SARS-COV-2 virus. Upon the development of the synthetic route of remdesivir, the selection of its S_{P} stereoisomer was favoured due to the easy crystallization of the S_{P} diastereomer precursor, as well as the better selectivity and wider therapeutic window of the target molecule. However, during the subsequent in vitro experiments it was revealed that both diastereomers possess adequate antiviral activity and that better efficacy of the R_{P}/S_{P} diastereomer depends on the type of tissue examined, which can be explained by different tissue distribution of the enzymes producing the biologically active form of remdesivir (Siegel et al., J. Med. Chem., 2017, 60, 1648-1661). According to this, the application of the R_{P} diastereomer may also be of clinical importance in the treatment of Covid-19 and other viral infections, therefore the development of asymmetric synthesis routes targeting the production of the R_{P} diastereomer and the realization of the separation of the R_{P}-S_{P} diastereomer mixture is of utmost importance.

The synthesis and purification of the synthesis intermediate and the final molecule have many challenges due to the presence of the numerous chirality centres in the molecule, especially the presence of the P-stereogenic centre which significantly affects the molecule's therapeutic activity. The difficult task of synthesizing the molecule with the appropriate configuration was successfully accomplished via the following main routes (Vargas et al., ACS Omega, 2021, 6, 19356-19363):

In the first total synthesis published by Gilead Sciences (US Patent No. U.S. Pat. No. 10,251,898B2) the 1:1 ratio diastereomeric mixture of the target molecule has been synthesized in a few steps, from which mixture the desired S_{P} diastereomer was extracted in a preparative chromatographic step using a Lux Cellulose-2 chiral column applying isocratic elution of acetonitrile/methanol (95/5, v/v). The selectivity of this expensive chiral separation was 1.45 and in an unfortunate way, the target S_{P} diastereomer eluted later, which rendered the efficacy and throughput of the separation low. In this patent, the separation of diastereomers has also been showcased on an analytical-scale Kinetex C18 column under achiral conditions using gradient elution, albeit with very low selectivity (<1.02). Overall, due to the several HPLC purification steps mid-synthesis and the final preparative chiral HPLC step, the scale-up of the first route was not feasible, and its inferior yield was also problematic.

In the second-generation synthesis route (WO 2017184668A1 published international application), for the development of a scalable synthetic route to produce gram-scale batches, several synthetic steps, such as the oxidative step required for the preparation of the key intermediate lactone or the C-glycosylation step have been modified and optimized using more economical reagents and milder reaction conditions, while also focusing on reaching better yields. The mild reaction conditions and the use of the enantiopure S_{P} isomer as a coupling agent enabled the diastereoselective synthesis of the final molecule, therefore the 200 g remdesivir required for preclinical testing could be produced in good yield, avoiding the expensive chromatographic steps. Kappe et al. also implemented the flow chemistry synthesis of the C-glycosylation step, which allowed for the substitution of the batch reaction step with a continuous step, with a nearly equivalent yield (Kappe et al., Org. Process Res. Dev. 2020, 24, 2113-2121.).

In the case of the first- and second-generation synthesis routes, it still remained a problem that neither route contained a reaction step which resulted in the stereoselective formation of the target molecule in the final step. To remedy this, Wang et al. developed a catalytic asymmetric step in which the last key step of the synthesis route, the coupling of the protected nucleoside and phosphoryl halide in the presence of a bicyclic imidazole catalyst, provided the S_{P} isomer of remdesivir with a 96% conversion and an over 20-fold diastereomeric excess. This reaction could also be scaled up to a scale of 10 grams, which foreshadowed the potential for its industrial application (Wang et al., Angew. Chem. Int. Ed., 2020, 10.1002/anie.202011527). Thinking alike, Ganedi et al. further improved the asymmetric catalytic synthesis of the phosphorus chirality centre after testing various bicyclic imidazole catalysts. By the means of their method, S_{P}-remdesivir could be produced with 95% purity and an over 40-fold diastereomeric excess (Gannedi et al., J. Org. Chem., 2021, 86, 4977-4985).

In contrast to the several described synthetic methods of S_{P}-remdesivir, there are only a few examples of synthetic routes for the production of the R_{P} diastereomer as the target compound. The synthesis of R_{P}-remdesivir can be accomplished using a Grignard reaction previously used for ProTide-type compounds, by coupling the key intermediate nucleoside and enantiopure R_{P} phosphoramidate (Yichen et al., Chinese J. Org. Chem., 2020, 40, 3237-3248). Bigley et al. produced the enantiopure phosphoramidate precursor needed for the synthesis of the R_{P}-isomer by a chemoenzymatic route using a stereoselective phosphotriesterase enzyme. From the enantiopure precursor, using the Grignard reaction described by Yichen et al. and coupling it with the nucleoside key intermedier, they were able to produce pure R_{P}-remdesivir on a milligram scale (Bigley et al., Biochemistry, 2020, 59, 3038-3043).

When the stereoselective synthesis cannot be realized, or when the reaction takes place with a poor enantiomeric/diastereomeric excess, it is also possible to extract the pure isomers of the compound from a mixture of the isomers using chromatographic separation. Besides classical solid-liquid chromatographic methods, liquid-liquid chromatography can also offer an alternative choice to this avail.

Liquid-liquid chromatography (LLC) is a unique preparative separation technique, which is considered as highly suitable for the purification of APIs, the separation of APIs from their impurities or to removal of unwanted (toxic) contaminations from biologically valuable matrices.

LLC methods have been successfully applied to separate the components of binary, structurally very similar compound mixtures that are difficult to separate even with classical chromatographic methods, typically molecules with some kind of chirality, such as enantiomers, and diastereomers (Duan et al., Chirality, 2020, 1-20; Tong et al., J. Chromatogr. A, 2020, 1626, 461345).

The chiral methods commonly used for the separation of enantiomers and diastereomers work on the principle of single-phase chiral recognition (one phase contains the chiral selector) or two-phase chiral recognition (both phases contain a selector each, which are only soluble in one phase), which results in different phase-distribution of the enantiomers/diastereomers to be separated. In the case of LLC, generally single-phase chiral recognition is used, mainly using cyclodextrin derivatives, chiral amino acid (L-proline) derivatives, tartaric acid derivatives as chiral selectors, but in some methods sugar derivatives, naproxen derivatives, human serum albumin, vancomycin, cinchona alkaloid derivatives and crown ethers have also been used. The different complex stability constants of the appropriate chiral selector and the association complexes of the different molecules to be separated result in the different distribution of the compound to be separated in the biphasic system, which is the key to the selectivity of the method. However, it is important to note here that, unlike enantiomeric pairs, diastereomeric molecules can be distinguished based on their polarity even in an achiral chemical environment without any chiral recognition (selector).

To further improve selectivity, the different and flexibly adaptable operating modes of the CPC technique provide an additional opportunity. The different LLC elution modes are discussed in detail in the review article published by Huang et al (Huang et al., Trends Analyt. Chem., 2016, 77, 214-225).

For the separation of compounds with very similar partition coefficients, such as enantiomers and diastereomers, especially when a chiral selector alone did not provide sufficient selectivity for the separation, the so-called multiple dual mode (MDM) can be used.

Rubio et al were able to separate the isomers of two racemic compounds (containing the two different isomers in a 50%-50% ratio) using the MDM mode in the presence of(S)-naproxen as a chiral selector. The S-isomer of naproxen facilitated the separation of one of the dinitrobenzamide derivatives in the presence of two-phase chiral recognition in the n-hexane/ethyl acetate/methanol/water solvent system, while in the case of the other derivatives, one-phase chiral recognition could be observed in the tert-butyl methyl ether/phosphate buffer (pH 6.0) solvent system. In their case, the CPC method, run in the classic elution mode, was not suitable for achieving the resolution required for the separation of the compounds, however, by choosing the optimal running parameters, baseline separation of the compounds was achieved in the MDM mode (Rubio et al., J. Chromatogr. A, 2009, 1216, 8505-8511).

Tong et al. also used MDM mode for the separation of enantiomers of aromatic carboxylic acid derivatives, in the presence of a hydroxypropyl-ß-cyclodextrin chiral selector. In the n-hexane/ethyl acetate/phosphate buffer (pH 2.67) solvent system containing 100 mM hydroxypropyl-ß-cyclodextrin, a significant increase in resolution was achieved using a modified MDM mode (Tong et al., J. Sep. Sci., 2013, 36, 2035-2042).

In another instance, Tong et al. separated naproxen enantiomers using the recycling elution mode. Choosing the optimal aqueous pH and hydroxypropyl-ß-cyclodextrin chiral selector concentration in the n-hexane/ethyl acetate/phosphate buffer (pH 2.67) solvent system, baseline separation of the enantiomeric pair was achieved after the third cycle (Tong et al., J. Chromatogr. A, 2011, 1218, 5434-5440).

Luca et al. used the trapping MDM mode to separate the minor cannabinoids of an industrial hemp extract, during which several minor components, including cannabielsoin, cannabidivarin, cannabigerol, cannabichromene and cannabicilol were successfully isolated using the two-phase systems of different ratios of n-hexane/methanol/water (Luca et al., J. Chromatogr. A, 2021, 1658, 462608).

As of now, liquid-liquid chromatography methods have not been applied for the separation of remdesivir diastereomers.

### SUMMARY OF THE INVENTION

The object of the present invention was to develop efficient, scalable, and cost-efficient separation methods by which the separation of Rₚ and S_{P} isomers of remdesivir can be realized. The inventors discovered that by applying liquid-liquid chromatographic methods and suitable solvent systems, the above goal can be achieved even in an achiral environment, without the use of a chiral selector. With the help of the developed method, in addition to isolating the S_{P} diastereomer, the difficult-to-produce and separate R_{P} isomer can also be isolated with high purity.

The object of the invention is therefore a method for the separation of phosphorus stereoisomers of remdesivir from an R_{P}-S_{P} remdesivir isomeric mixture by liquid-liquid chromatography (LLC) using a biphasic, binary, ternary or quaternary solvent system, wherein
the binary solvent system consists of the following phase-forming components:
   an alkanoate ester with 3-6 carbon atoms; and
   an aqueous solution, containing a solubility-enhancing additive and a buffer salt and/or an organic acid used for setting the pH of the aqueous phase in the range of pH 2-7;
the ternary solvent system consists of the following phase-forming components:
   an aliphatic alkane with 5-8 carbon atoms;
   a furane-type aromatic heterocyclic compound or an alkanoate ester with 3-6 carbon atoms;
   an aqueous solution, containing a solubility-enhancing additive and/or a buffer salt and/or an organic acid used for setting the pH of the aqueous phase in the range of pH 2-7;
the quaternary solvent system consists of the following phase-forming components:
   an aliphatic alkane with 5-8 carbons;
   a furane-type aromatic heterocyclic compound;
   a further organic co-solvent solvent selected from an aliphatic alcohol with 2-5 carbons, preferably methanol or ethanol; a ketone with 2-4 carbons, preferably acetone; an organic nitrile with 2-3 carbons, preferably acetonitrile; dimethyl sulfoxide; N,N-dimethylformamide; N,N-dimethylacetamide; and 1,2-dimethoxyethane;
   water or an aqueous solution containing solubility-enhancing additive and/or a buffer salt and/or an organic acid used for setting the pH of the aqueous phase in the range of pH 2-7.

The alkanoate ester is preferably ethyl acetate, n-propyl acetate, isopropyl acetate or n-butyl acetate.

The furane-type aromatic heterocyclic compound is preferably 2-methyltetrahydrofuran.

The solubility-enhancing additive is preferably a cyclodextrin or its derivative or a polyethylene glycol, preferably sulfobutylether-ß-cyclodextrin, preferably polyethylene glycol-1500.

The buffer salt is preferably citrate or phosphate buffer salt; and the organic acid is preferably formic acid, acetic acid or trifluoroacetic acid.

The LLC separation can be realized in hydrodynamic (counter-current chromatography, CCC) or in hydrostatic (centrifugal partition chromatography, CPC) device, preferably in a dual-rotor CPC device.

Preferably the following solvent systems are applied: ethyl acetate/100 mM sulfobutylether-ß-cyclodextrin, pH 2.0 phosphate buffer in a 1/1 (v/v) ratio; n-hexane/ethyl acetate/100 mM sulfobutylether-ß-cyclodextrin, pH 3.0 citrate buffer in a 1/1/1 (v/v/V) ratio; or n-heptane/2-methyltetrahydrofuran/N,N-dimethylformamide/water in a 3/6/1.3/4 (v/v/v/v) ratio.

According to one preferred embodiment the LLC is performed in classical elution mode using descending or ascending mode. In another preferred embodiment the LLC is performed in trapping MDM mode, where the isomer mixture is dissolved in both phases of the solvent system and injected between the two LLC columns according to the elution mode: during ascending run the upper phase, while during descending run the lower phase is injected.

According to a further preferred embodiment the LLC is performed in MDM mode, where the isomer mixture is dissolved in either the upper or the lower phase of the solvent system and injected between the two columns of the LLC instrument followed by elution in MDM mode.

During the process preferably collecting the adequate product fractions, >95% pure R_{P} and/or S_{P} isomer(s) is/are recovered.

### BRIEF DESCRIPTION OF THE FIGURES

FIG. 1 shows the HPLC-UV chromatograms of the isomeric mixtures used during the LLC separations (FIG. 1/A and 1/B), and the isolated R_{P} remdesivir product of Example 1 (FIG. 1/C, purity: 99.0%) (evaluation: 220±4 nm)

FIG. 2 shows the 31P NMR spectra of the isomeric mixtures used during the LLC separations (FIG. 2/A and 2/B), and the isolated R_{P} remdesivir product of Example 1 (FIG. 2/C, purity: 99.0%) (Instrument: Bruker Avance III HD 600, Prodigy with cryoprobe; 31P: 242.89 MHZ, solvent: CDCl₃, temperature: 295 K, internal standard: TMS)

FIG. 3 shows the fractograms of CPC runs performed in classical ascending (A) and descending (B) elution modes in the n-heptane/2-methyltetrahydrofuran/N,N-dimethylformamide/water 3/6/1.3/4 (v/v/v/v) solvent system at 50% stationary phase retention on a laboratory-scale CPC instrument.

FIG. 4 shows the offline reconstructed fractograms of a laboratory-scale CPC separation run in trapping multiple dual mode (MDM) for the separation of remdesivir isomeric mixture containing 24.14% R_{P} isomer and 75.70% S_{P} isomer in the n-heptane/2-methyltetrahydrofuran/N,N-dimethylformamide/water 3/6/1.3/4 (v/v/v/v) solvent system under the experimental conditions described in Example 1 and Table 4.

FIG. 5 shows the chromatogram of the separation of the isomeric mixture of remdesivir containing 24.14% R_{P} isomer and 75.70% S_{P} isomer in the n-hexane/ethyl acetate/100 mM SBECD, pH 3.0 citrate buffer 1/4/5 (v/v/v) solvent system on a laboratory-scale CPC in MDM mode applying dsc-asc-dsc operating modes under the experimental conditions described in Example 2 and Table 5.

FIG. 6 shows the fractogram of the separation of the isomeric mixture of remdesivir containing 24.14% R_{P} isomer and 75.70% S_{P} isomer in the n-hexane/ethyl acetate/100 mM SBECD, pH 3.0 citrate buffer 1/4/5 (v/v/v) solvent system on a laboratory-scale CPC in MDM mode applying dsc-asc-dsc operating modes under the experimental conditions described in Example 2 and Table 5.

FIG. 7 shows the chromatogram of the separation of the isomeric mixture of remdesivir containing 43.18% R_{P} isomer and 56.12% S_{P} isomer in the ethyl acetate/100 mM SBECD, pH 2.5 phosphate buffer 1/1 (v/v) solvent system on a laboratory-scale CPC in MDM mode applying dsc-asc-dsc-asc-dsc operating modes under the experimental conditions described in Example 3 and Table 6.

FIG. 8 shows the fractogram of the separation of the isomeric mixture of remdesivir containing 43.18% R_{P} isomer and 56.12% S_{P} isomer in the ethyl acetate/100 mM SBECD, pH 2.5 phosphate buffer 1/1 (v/v) solvent system on a laboratory-scale CPC in MDM mode applying dsc-asc-dsc-asc-dsc operating modes under the experimental conditions described in Example 3 and Table 6.

FIG. 9 shows the chromatogram of the separation of the isomeric mixture of remdesivir containing 24.14% R_{P} isomer and 75.70% S_{P} isomer in the n-heptane/2-methyltetrahydrofuran/N,N-dimethylformamide/water 3/6/1.3/4 (v/v/v/v) solvent system on a laboratory-scale CPC in MDM mode applying dsc-asc-dsc-asc-dsc-asc operating modes under the experimental conditions described in Example 4 and Table 7.

FIG. 10 shows the fractogram of the separation of the isomeric mixture of remdesivir containing 24.14% R_{P} isomer and 75.70% S_{P} isomer in the n-heptane/2-methyltetrahydrofuran/N,N-dimethylformamide/water 3/6/1.3/4 (v/v/v/v) solvent system on a laboratory-scale CPC in MDM mode applying dsc-asc-dsc-asc-dsc-asc operating modes under the experimental conditions described in Example 4 and Table 7.

FIG. 11 shows the chromatogram of the separation of the isomeric mixture of remdesivir containing 43.18% R_{P} isomer and 56.12% S_{P} isomer in the n-heptane/2-methyltetrahydrofuran/N,N-dimethylformamide/water 3/6/1.3/4 (v/v/v/v) solvent system on a laboratory-scale CPC in MDM mode applying dsc-asc-dsc operating modes under the experimental conditions described in Example 5 and Table 8.

FIG. 12 shows the fractogram of the separation of the isomeric mixture of remdesivir containing 43.18% R_{P} isomer and 56.12% S_{P} isomer in the n-heptane/2-methyltetrahydrofuran/N,N-dimethylformamide/water 3/6/1.3/4 (v/v/v/v) solvent system on a laboratory-scale CPC in MDM mode applying dsc-asc-dsc operating modes under the experimental conditions described in Example 5 and Table 8.

FIG. 13 shows the chromatogram of the separation of the isomeric mixture of remdesivir containing 43.18% R_{P} isomer and 56.12% S_{P} isomer in the n-heptane/2-methyltetrahydrofuran/N,N-dimethylformamide/water 3/6/1.3/4 (v/v/v/V) solvent system on a pilot-scale CPC in MDM mode applying dsc-asc-dsc operating modes under the experimental conditions described in Example 6 and Table 9.

FIG. 14 shows the fractogram of the separation of the isomeric mixture of remdesivir containing 43.18% R_{P} isomer and 56.12% S_{P} isomer in the n-heptane/2-methyltetrahydrofuran/N, N-dimethylformamide/water 3/6/1.3/4 (v/v/v/v) solvent system on a pilot-scale CPC in MDM mode applying dsc-asc-dsc operating modes under the experimental conditions described in Example 6 and Table 9.

### DETAILED DESCRIPTION OF THE INVENTION

The invention relates to the separation of Rₚ and S_{P} isomers of remdesivir, which contain ligands in different spatial arrangements around the phosphorus atom, using liquid-liquid chromatography (LLC) to obtain the S_{P} isomer and/or the currently difficult-to-obtain R_{P} diastereomer with the highest possible purity (typically above 95% purity) and good production yield from the mixture of its isomers.

In general, it can be stated that the prerequisite for a successful LLC separation is the different distribution of the components in the phases of the given solvent system (distribution coefficients (K_{d})), from which the selectivity of the method (quotient of K_{d} values) also arises. In liquid-liquid chromatography (LLC) both the stationary and mobile phases are liquids and separation is based on the partitioning of the solutes between these immiscible liquid phases. LLC methods can be realized in hydrodynamic (called Counter-Current Chromatography, CCC) and in hydrostatic (called Centrifugal Partition Chromatography) devices and a given LLC method is perfectly transferable between any CCC and CPC equipment. A general description of the LLC technique can be found in the book Countercurrent Chromatography—The Support-Free Liquid Stationary Phase” Billardello, B.; Berthod, A; Wilson & Wilson's Comprehensive Analytical Chemistry 38; Berthod, A., ed.; Elsevier Science B.V.: Amsterdam (2002).

In the hydrostatic-type realization of LLC—called Centrifugal Partition Chromatography (CPC)—a series of cells interconnected by ducts are attached to a rotor and filled with the liquid stationary phase, which is immobilized inside the rotor by a strong centrifugal force. The other phase of the two-phase solvent system is used as the mobile phase containing the sample to be purified: it is fed under pressure into the rotor and pumped through the stationary phase in the form of tiny droplets (i.e., percolation). An important feature of CPC is that the direction of the flow and role of the phases are interchangeable: CPC operates in ascending (asc) mode when the lower (denser) phase of the biphasic solvent system is used as stationary phase, while the CPC experiment is performed in descending (dsc) mode when the upper (lighter) phase is immobilized and used as stationary phase.

Because retention and thus selectivity in CPC is governed solely by partitioning, the rate at which each compound (solute) travels through the system is fully predictable once the compound's partition coefficient (K_{d}) is known. Determination of the K_{d} value is performed routinely by the shake-flask method during method optimization and by convention expressed as the ratio of the concentration of the compound in the upper phase to its concentration in the lower phase. As a rule of thumb, K_{d} of the targeted compounds in an optimal solvent system should preferably be in the range of 0.5-2.0 (the so-called sweet spot), where the resolving power of CPC is considered as maximum.

Note that in the case of inverting the role of the liquid phases (asc to dsc or dsc to asc), the reciprocal values of the corresponding K_{d} values should be considered. In a broader sense, however, the given separation can also be realized in a solvent system with a K_{d} value between 0.25 and 4.0. It is important to note that when changing the roles of the liquid phases (from ascendant to descendent or vice versa), the reciprocal values of the relevant K_{d} values must be calculated. This characteristic therefore provides the LLC technique with the possibility to change the retention order of the compounds to be separated, which cannot be realized in solid-charge chromatography or can only be realized with great difficulty.

In the present invention, the R_{P} and S_{P} diastereomers of remdesivir are obtained by a liquid-liquid chromatography (LLC) method from an isomeric mixture containing R_{P} and S_{P} remdesivir, using a two-phase solvent system for the separation. The mixture of isomers is a physical mixture containing the R_{P} and S_{P} diastereomers of remdesivir in the main mass (typically >99% (w/w)) in any ratio.

The components of the applied two-phase solvent systems were chosen in such a way that they contain components that dissolve the compounds to be separated well, as well as components that dissolve the compounds moderately or not at all, which are thus jointly responsible for the selectivity stemming from the concurrent presence of attractive and repulsive interactions. Based on our recognition, it was not necessary to form a chiral interaction (recognition) for a successful separation, therefore we did not use a costly chiral selector, which allowed the optimization of the solvent systems in an achiral chemical environment. Cyclodextrins were an exception to this, however, they were used solely for their water solubility-enhancing effect since it has been revealed that they did not increase selectivity between the remdesivir P-isomers.

When selecting the components of the two-phase solvent system applicable for the separation of remdesivir isomers, we considered that the remdesivir isomers to be separated are practically insoluble in water, alkanes, aromatic hydrocarbons (e.g. toluene, xylene, trifluorotoluene), and aliphatic ethers (<10 mg/mL), are sparingly soluble in isopropanol, butanol, isopropyl and butyl acetate and alcohols and esters with longer carbon chain, methyl-isobutyl-ketone, trifluoroethanol, benzyl alcohol, morpholine, N-methyl pyrrolidone, nitromethane and cyrene (10-150 mg/mL), their solubility is moderately good in ethanol, 2-butoxyethanol, methyl and ethyl acetate, propylene glycol methyl ester, acetone, acetonitrile, tetrahydrofuran, 2-methyltetrahydrofuran, chloroform and dichloromethane (150-250 mg/mL) and excellent in methanol, 2-methoxyethanol, 1,2-dimethoxyethane, dimethyl sulfoxide and 2-butanon (>250 mg/ml).

When looking for a solvent system, we kept in mind that the K_{d} value of the isomers to be separated should ideally be in the optimal range of 0.5-2.0, but it must definitely fall within the still feasible range of 0.25-4.0 (Table 1). Since, in the case of the LLC technique, the retention is only affected by the distribution, the distribution coefficients clearly determine the chromatographic elution profile, therefore their knowledge is sufficient to predict the goodness of the separation. In addition, we had to consider the selectivity required for successful separation (classical elution method: α>1.4; DM or MDM mode: 1.2<<<1.4) (Table 1). Based on these aspects, we searched for solvent systems suitable for CPC separation, in which the conditions for sufficient solubility in both phases, optimal distribution coefficients and appropriate selectivity are met at the same time.

TABLE 1
Distribution coefficients of R_{P} and S_{P} diastereomers of remdesivir in solvent systems with different
compositions suitable for LLC seperation (*: suitable for MDM and trapping MDM, suitable only with
low yields for classical elution mode; ** suitable for MDM and trapping MDM and classical elution mode) at
room temperature. Using the solvent sytem marked with ***, efficient seperation cannot be realized due to
high K_{d} values, its inclusion in the table is meant to showcase the trend recognized during method
development. SBECD: sulfobutylether-β-cyclodextrin; PEG-1500: polyethylene glycol 1500.

| | | | Distribution | | |
|---|---|---|---|---|---|
| | | | coefficient (K_{d}) | | |
| | | Composition | R_{P} | S_{P} | Selectivity |
| Nr. | Solvent system | (v/v) | isomer | isomer | (α) |
| 1 | ethyl acetate/100 mM SBECD, pH 2.5 phosphate buffer | 1/1 | 1.16 | 1.42 | 1.22* |
| 2 | n-propyl acetate/100 mM SBECD, pH 2.0 phosphate | 1/1 | 0.44 | 0.56 | 1.28* |
| | buffer | | | | |
| 3 | n-butyl acetate/250 mM SBECD, pH 3.8 citrate buffer | 1/1 | 1.22 | 1.50 | 1.27* |
| 4 | n-hexane/ethyl acetate/100 mM SBECD, pH 3.0 citrate | 1/4/5 | 1.12 | 1.42 | 1.23* |
| | buffer | | | | |
| 5*** | n-heptane/2-methyltetrahydrofuran/water | 1/1/1 | 6.08 | 9.23 | 1.52 |
| 6 | n-heptane/2-methyltetrahydrofuran/water 200 mM | 1/1/1 | 1.77 | 2.60 | 1.47** |
| | SBECD | | | | |
| 7 | n-heptane/2-methyltetrahydrofuran/pH 2.5 citrate buffer | 1/1/1 | 1.26 | 1.63 | 1.29* |
| 8 | n-heptane/2-methyltetrahydrofuran/methanol/pH 4.0 | 1/1/0.3/1 | 1.41 | 2.02 | 1.43** |
| | citrate buffer | | | | |
| 9 | n-heptane/2-methyltetrahydrofuran/methanol/water | 1/1/0.15/1 | 2.62 | 3.92 | 1.50** |
| 10 | n-heptane/2-methyltetrahydrofuran/methanol/water | 1/1/0.3/1 | 1.16 | 1.65 | 1.43** |
| 11 | n-hexane/2-methyltetrahydrofuran/methanol/water | 1/1/0.3/1 | 0.78 | 1.01 | 1.30* |
| 12 | isooctane/2-methyltetrahydrofuran/methanol/water | 1/1/0.3/1 | 0.97 | 1.31 | 1.36* |
| 13 | cyclohexane/2-methyltetrahydrofuran/methanol/water | 1/1/0.3/1 | 1.90 | 2.64 | 1.39* |
| 14 | n-heptane/2-methytetrahydrofuran/ethanol/water | 1/1/0.3/1 | 1.53 | 2.05 | 1.34* |
| 15 | n-heptane/2-methytetrahydrofuran/dimethyl | 1/1/0.3/1 | 1.13 | 1.74 | 1.54** |
| | sulfoxide/water | | | | |
| 16 | n-heptane/2-methytetrahydrofuran/acetonitrile/water | 1/1/0.3/1 | 2.51 | 3.37 | 1.35* |
| 17 | n-heptane/2-methyltetrahydrofuran/acetone/water | 1/1/0.3/1 | 2.16 | 3.09 | 1.44** |
| 18 | n-heptane/2-methyltetrahydrofuran/1,2- | 1/1/0.3/1 | 2.65 | 3.91 | 1.48** |
| | dimethoxyethane/water | | | | |
| 19 | n-heptane/2-methyltetrahydrofuran/2- | 1/1/0.3/1 | 1.15 | 1.62 | 1.41** |
| | metoxiethanol/water | | | | |
| 20 | n-heptane/2-methyltetrahydrofuran/N,N- | 3/6/1.3/4 | 1.54 | 1.99 | 1.29* |
| | dimethylformamide/water | | | | |
| 21 | n-heptane/2-methyltetrahydrofuran/N,N- | 3/6/1.3/4 | 1.04 | 1.36 | 1.31* |
| | dimethylacetamide/water | | | | |
| 22 | n-heptane/2-methyltetrahydrofuran/methanol/0.5% | 1/1/0.06/1 | 1.11 | 1.41 | 1.27* |
| | (v/v %) formic acid in water | | | | |
| 23 | n-heptane/2-methytetrahydrofuran/methanol/20% | 1/1/0.06/1 | 0.75 | 1.04 | 1.38* |
| | (w/w %) PEG-1500 in water | | | | |
| 24 | n-heptane/2-methytetrahydrofuran/methanol/200 mM | 1/1/0.06/1 | 0.49 | 0.63 | 1.29* |
| | SBECD | | | | |

The present invention can be executed using binary, ternary and quaternary two-phase solvent systems.

In the case of binary system, suitable systems were identified via simultaneous optimization of the pH of the aqueous phase, and the solubility-enhancing additive (e.g., cyclodextrin or PEG) content of the system (solvent systems #1-3 in Table 1). In the case of ternary systems, the organic phase of suitable systems consists of an alkane and beside the alkane an ester or furan promoting the partition of remdesivir into the upper phase. The suitable ternary systems were identified via simultaneous optimization of the pH of the aqueous phase and the solubility-enhancing additive (e.g., cyclodextrin or PEG) and co-solvent content of the system (solvent systems #4-7 in Table 1). The suitable quaternary systems were identified by an analogous optimization process as in the ternary case after the addition of a further organic co-solvent to the system that promoted the partition of remdesivir into the lower aqueous phase (solvent systems #8-24 in Table 1). In some cases, aqueous solution containing no solubility-enhancing additives also proved to be suitable amongst the quaternary systems (solvent systems #9-21 in Table 1).

Suitable binary solvent systems consist of an organic ester (alkanoate ester with 3-6 carbon atoms), a solubility-enhancing additive and a buffering agent to maintain the pH of the aqueous phase in the range of pH 2-7.

By aqueous solubility-enhancing additive, we mean excipients that significantly (preferably at least by 50%) increase the aqueous solubility of remdesivir isomers, such as e.g., various natural cyclodextrins (alpha-, beta- or gamma cyclodextrin) and their derivatives (preferably sulfobutylether-ß-cyclodextrin) or polyethylene glycols (PEGs) with different degree of polymerization (preferably PEG-1500).

The pH of the aqueous phase of the suitable solvent systems considering the chemical stability of remdesivir must necessarily fall within the pH 2.0-7.0 range, while based on the maximum of solubility of remdesivir-considering the ionization constant of remdesivir (basic pkₐ=3.56; Várnai et al., J. Pharm. Biomed. Anal., 2021, 209, 114482)—the preferable range is between pH 3.5-4.0.

To maintain the pH in the suitable range, a buffer salt possessing buffer capacity in the pH range of pH 2-7, preferably citrate or phosphate salt or an organic acid can be used. By “organic acid”, we mean certain carboxylic acids, widely used as acidic modifiers in chromatographic practice, whose pH value falls below 3 in a 0.1-1.0% (v/v) aqueous solution. Preferred examples of this are formic acid, acetic acid, and trifluoroacetic acid.

Ternary and quaternary solvent systems were composed using alkanes with 5-8 carbon atoms (e.g., pentane, cyclohexane, isooctane, preferably hexane or heptane) as hydrophobic organic component, a second organic solvent that dissolves remdesivir well, such as a furan-type heterocyclic aromatic solvent (e.g., 2-methyltetrahydrofuran) and in case of ternary solvent systems it further may be an alkanoate ester (preferably ethyl acetate, propyl acetate, isopropyl acetate, or n-butyl acetate), while in the case of quaternary solvent systems a third organic co-solvent, such as an aliphatic alcohol with 2-5 carbon atoms (e.g., methanol or ethanol), or a ketone with 2-4 carbon atoms (e.g., acetone), or an organic nitrile (e.g., acetonitrile) or dimethyl sulfoxide, or N,N-dimethylformamide, or N,N-dimethyl acetamide, or 1,2-dimethoxy ethane have been applied.

We have found that in the case of ternary solvent systems, it might be sufficient for achieving satisfactory separation if the aqueous solution contains only one type of the additives, i.e., a water solubility-enhancing additive or a buffer salt to maintain pH.

In the case of quaternary solvent systems, upon addition of a further organic co-solvent even both additives might be omitted, therefore solvent systems containing only pure water in the aqueous phase might also be used.

The preferred solvent systems are characterized by a short settling time (less than 30 sec), a good dissolving ability (more than 20 mg racemic mixture of remdesivir can be dissolved in 1 mL of the solvent system), while from the toxicological aspect, their components can be categorized into class 3 and/or class 4 solvents classes.

The LLC separation can be realized in hydrodynamic (counter-current chromatography, CCC) or in hydrostatic (centrifugal partition chromatography, CPC) device.

The separation can be carried out using different LLC modes, which-knowing the distribution coefficient of the compounds to be separated—can be properly selected and optimized by a person skilled in the art.

When implementing the simplest mode, the so-called “classical elution mode”, the elution and separation of the components of the sample are realized by pumping the mobile phase through the stationary phase in either ascending or descending direction after the sample is injected onto the rotor. In the case of structurally very similar compounds, such as the remdesivir isomers, those systems are considered suitable for separation in classical elution mode, where selectivity (α=K_{d1}/K_{d2}, where K_{d1} is the larger distribution coefficient, while K_{d2} is the smaller distribution coefficient) reaches the value of 1.4 for the isomeric pairs. In this case, in a liquid-liquid chromatographic system applying a stabile (rapidly settling) solvent system that provides 50% stationary phase retention, on a column that operates with at least 500 theoretical plate number, a good separation with decent yields can be achieved. When reaching a separation factor at least this high, separation using liquid-liquid chromatography methods can be superior to even high-performance chromatographic methods.

If the desired separation factor cannot be achieved this way-only reaches a value between 1.2 and 1.4-instead of the classical elution mode further, flexibly adaptable so-called advanced operating modes of LLC can be selected to increase resolution. A typical case of this is when the valuable components are eluted in the optimal elution window, but their resolution is not sufficient: in this case, exploiting the fact that in liquid-liquid chromatographic systems both the mobile and stationary phases are liquid, and thus their roles are interchangeable during the same run by reversing their role and the direction of their flow (from ascending to descending or vice versa) during the separation, a higher number of plates can be achieved in the chromatographic system. This so-called dual mode can be typically used in the case of complex sample matrices containing components with a wide range of K_{d} values. Operation in a simple dual mode is often not sufficient for the separation of compounds with close partition coefficients, such as enantiomers and diastereomers, therefore the application of the so-called multiple dual mode (MDM) may be necessary. In this case, a series of switches typical in dual mode are executed reversing the direction of the flow of the phases each time, while keeping the compound to be separated inside the column until a satisfactory separation can be achieved. In such cases, the peak resolution can theoretically be improved by increasing the number of theoretical plates in the chromatographic system. However, in practice, the increase in the number of theoretical plates achieved this way is also affected by the extent of diffusion inside the column, therefore, the optimization of the number of cycles and critical switching times (time periods for switching between the ascending and descending operation modes) and the collection time of fractions is pivotal for finding the optimal operating parameters that allow for achieving the best possible resolution and purity (Delannay et al., J. Chromatogr. A, 2006, 1127, 45-51). To facilitate the determination of optimal parameters for separations run in the different modes, Kostanyan et al. performed the mathematical description of the different systems, by means of which the effect of parameters on the separation can be modelled without performing the actual DM/MDM mode runs. (Kostanyan et al., Molecules, 2020, 25, 6020).

For the separation of remdesivir stereoisomers chromatographic runs in MDM mode are preferably implemented when the selectivity (a) is between 1.2 and 1.4.

The possibility of reversing the roles of the stationary and mobile phases and the direction of the flow is also exploited in the so-called sequential CPC (sCPC) mode (Minceva et al., Chem. Eng. Technol., 2012, 35, 72-82). This mode of operation can also be divided into cycles, during which the mixture to be separated is continuously injected onto the columns between the two columns (rotors), while the separated compounds are continuously collected on the opposite ends of the rotors. In the case of a two-component mixture when component A partitions rather into the lower phase, while component B partitions rather into the upper phase, the operating process can be described as follows: every cycle consists of two steps, in which the direction of the flow and the role of the upper and lower phases are once reversed simultaneously. At the beginning the rotors are filled with a 50:50% (v/V %) mixture of the stationary and mobile phases, then in the first, for example descending (dsc) step we start to pump the mobile phase at the end of the first column, meanwhile, the lower phase containing the sample is injected between the two columns using a second pump. In this setup, component A travels faster through the column, thus we can collect it at the end of the second column. In the next step, the direction of the flow is reversed and the mobile phase is pumped from the end of the second column, which this time becomes the upper phase according to the ascending (asc) operating mode, meanwhile, the sample is injected between the two columns dissolved in the upper phase. In this case we can expect a faster migration of component B, thus it can be collected at the end of the first column. In this operational mode, the method can be optimized by changing the flow rate of the mobile phase and sample feeding and varying the duration of the ascending and descending sections.

A special case of the sCPC method is the so-called trapping MDM mode, which can be applied for the separation and purification of one component of a multi-component mixture with an intermediate retention time in the classical elution mode (Minceva et al, J. Chromatogr. A, 2017, 1501, 26-38). In this case, the sample is injected between two columns, similar to the sCPC mode, and then, using ascending and descending cycles, sample components that elute earlier and later than the target component are collected at the two ends of the two rotors, meanwhile the target component remains “trapped” on the column until the end of the LLC run. This method consists of three main stages, which can be described for three components (A—the component eluting before the target component, B—the target component, C—the component eluting after the target component) as follows: a single loading cycle (cycle 0), during which both columns are filled, and then a part of the sample mixture dissolved in the lower phase is injected to the first rotor between the two rotors in descending mode, while the other part of the sample dissolved in the upper phase is injected to the other column between the two rotors in ascending mode. During the second, separation stage, components A and C are collected alternately in descending and ascending mode in subsequent cycles (cycle 1-cycle N) at one and the other end of the system, while component B is trapped on the columns. In the third, product recovery stage, after the components A and C eluting before and after the sample have been removed from the sample, the target compound B, which was retained on the column until now can be eluted in its purified from in ascending or descending mode. In order to achieve the maximum throughput of the method, it is necessary to optimize the maximum feed concentration and feed flow rate, and the time of the filling in cycle 0, and the time of the ascending and descending sections in cycles 1-N, as well as the number of cycles (N). Similar to the sCPC method, a mathematical model of the process can be implemented to optimize the parameters of the trapping MDM run, thus the optimized parameters can be obtained in using computer simulation after minimal experimental work. The steps and validation of this simulation were presented in detail by Goll et al (Goll et al, J. Chromatogr. A, 2017, 1496, 68-79).

For the separation of remdesivir stereoisomers chromatographic runs in trapping MDM mode are preferably implemented, when the selectivity (a) is between 1.2 and 1.4 and the target component is treated as component B, while the other isomer is treated as impurity A or C depending on its distribution coefficient during the simulation of the method.

According to the present invention, the LLC method can be executed in (i) classical elution mode; (ii) trapping multiple dual mode; or (iii) multiple dual mode.

In one embodiment of the method, presented in detail in the examples below, the R_{P}-S_{P} remdesivir isomeric mixture was separated on a laboratory-scale instrument, in classical elution mode and then in trapping MDM mode, after determining the optimal operating parameters by computer simulation.

In three other embodiments of the method R_{P}-S_{P} remdesivir isomeric mixtures with two different isomeric composition were separated using three different solvent systems on a laboratory-scale instrument, after the experimental determination of the optimal operating parameters.

In another embodiment of the method, scaling up the laboratory-scale method the R_{P}-S_{P} remdesivir isomeric mixture was separated in MDM mode using a pilot-scale CPC instrument, after the experimental determination of the optimal operating parameters. With the different embodiments of the method in each case, the R_{P} and/or S_{P} isomer of remdesivir was/were obtained with high purity, i.e., at least 95% (w/w), preferably 99% (w/w) from the isomeric mixtures, with medium or good yields.

### EXAMPLES

The following examples are provided by way of illustration of the invention and do not intend to restrict the scope of the invention as defined in the claims. Table 2 details six preferred embodiments of the invention on three different CPC devices from three different manufacturers (Armen, Gilson, and RotaChrom) with two different cell geometry (spherical and cylindrical cells) on laboratory-scale (2×90.1 ml and 2×250 ml rotor volumes) and on pilot-scale (2×2.1 L rotor volumes), respectively. Table 3 summarizes the purity, yield, and productivity values for the R_{P} and S_{P} isomers obtained with the six preferred embodiments of the invention.

TABLE 2
Summary of the main parameters of the laboratory- and pilot-scale CPC methods implemented in Examples 1-6.

| | | | | Composition | | | | | | |
|---|---|---|---|---|---|---|---|---|---|---|
| | | | | of starting | | Sample | | | | |
| | CPC | | | sample (S_{P} | | solvent | | | | |
| | device, | | Ratio of | and R_{P} | | and | | Rotational | Volumetric | |
| | rotor | Solvent | components | isomeric | Technique | solvent of | Loading | speed | flow | Runtime |
| Example | volume | system | (v/v) | ratio) | and mode | injection | (mg) | (1/min) | (mL/min) | (min) |
| 1 | Armen | n-heptane/ | 3/6/1.3/4 | 24.14% R_{P}, | classical | UP-LP | 25 | 1700 | 10 | 45 |
| | SCPC- | 2-MeTHF/ | | 75.70% S_{P} | elution, | | | | | |
| | 250 | DMF/water | | | dsc | | | | | |
| | 182.1 mL | | | | mode | | | | | |
| | | | | | trapping | UP-LP | 276 | 1700 | 10 | 177.5 |
| | | | | | MDM | | | | | |
| 2 | Gilsom | n- | 1/4/5 | 24.14% R_{P}, | MDM | UP | 150 | 1800 | 30 | 45 |
| | CPC | hexane/ethyl | | 75.70% S_{P} | | | | | | |
| | 2 × 250 mL | acetate/100 | | | | | | | | |
| | | mM SBECD | | | | | | | | |
| | | pH 3.0 citrate | | | | | | | | |
| | | buffer | | | | | | | | |
| 3 | Gilson | ethyl | 1/1 | 43.18% R_{P}, | MDM | UP | 150 | 1800 | 30 | 106 |
| | CPC | acetate/100 | | 56.12% S_{P} | | | | | | |
| | 2 × 250 mL | mM SBECD | | | | | | | | |
| | | pH 2.5 | | | | | | | | |
| | | phosphate | | | | | | | | |
| | | buffer | | | | | | | | |
| 4 | Gilson | n-heptane/ | 3/6/1.3/4 | 24.14% R_{P}, | MDM | UP | 250 | 2000 | 30 | 90 |
| | CPC | 2-MeTHF/ | | 75.70% S_{P} | | | | | | |
| 5 | 2 × 250 mL | DMF/water | | 43.18% R_{P}, | MDM | UP | 250 | 2000 | 30 | 50 |
| | | | | 56.12% S_{P} | | | | | | |
| 6 | RotaChrom | n-heptane/ | 3/6/1.3/4 | 43.18% R_{P}, | MDM | UP | 2000 | 1000 | 200 | 52 |
| | rCPC | 2-MeTHF/ | | 56.12% S_{P} | | | | | | |
| | 2 × 2.1 L | DMF/water | | | | | | | | |

UP: the upper phase of the solvent system; LP: the lower phase of the solvent system; 2-MeTHF: 2-methyltetrahydrofuran; DMF: N,N-dimethylformamide; MDM: multiple dual mode.

TABLE 3
Purity, yield and productivity values for the R_{P} and S_{P} isomers of remdesivir
obtained in Examples 1-6 using laboratory-scale and pilot-scale CPC methods.

| | R_{P} isomer | | | | | S_{P} isomer | | | |
|---|---|---|---|---|---|---|---|---|---|
| | Technique | Combined | purity | yield | productivity | Combined | purity | yield | productivity |
| Example | and mode | fractions | (%) | (%) | (mg/L_{column}/h) | fractions | (%) | (%) | (mg/L_{column}/h) |
| 1 | classical | — | — | — | — | 17-27 | 98.4 | 36.6 | 51.3 |
| | elution, dsc | | | | | | | | |
| | mode | | | | | | | | |
| | trapping | 1-34 | 95.1 | 54.5 | 126 | — | — | — | — |
| | MDM | 1-22 | 99.0 | 38.6 | 89.6 | — | — | — | — |
| 2 | MDM | — | — | — | — | 11-14, 42-53 | 95.7 | 53.8 | 170.2 |
| 3 | MDM | 71-88 | 95.8 | 19.2 | 14.7 | 12-13, 39-40 | 93.1 | 18.5 | 19.0 |
| | | | | | | 119-133 | | | |
| 4 | MDM | 69-80, | 95.5 | 72.3 | 78.3 | 25-28, 52-55, | 97.8 | 92.0 | 305.4 |
| | | 99-102 | | | | 89-96 | | | |
| 5 | MDM | 46-60 | 95.6 | 81.5 | 220.7 | 25-28, 65-72 | 95.7 | 85.3 | 300.2 |
| 6 | MDM | 6-22 | 95.0 | 65.4 | 169.2 | 30-33, 84-92 | 95.1 | 58.3 | 195.7 |

### Example 1

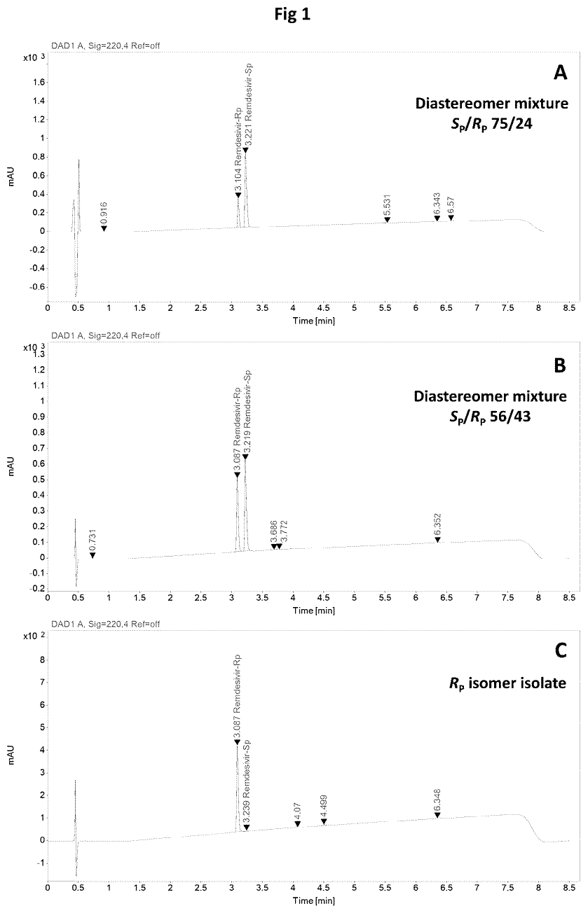

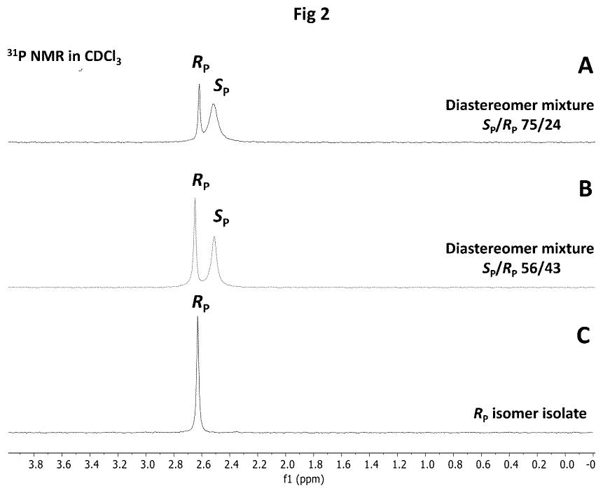

The aim of the experiment was the separation of phosphorus stereoisomers of remdesivir to obtain high purity R_{P} isomer in trapping MDM mode, on a laboratory-scale dual-rotor CPC device. During the method development of the trapping MDM run, the isolation of the S_{P} isomer was demonstrated in classical elution mode. The remdesivir isomer mixture to be separated was obtained commercially from Omgene Life Sciences. Based on HPLC/DAD/MS measurements it consisted of 24.14% R_{P} isomer, 75.70% S_{P} isomer and 0.16% other impurity (HPLC chromatogram: FIG. 1/A, ³¹P-NMR spectra: FIG. 2/A).

The experiment was carried out on an Armen SCPC-250 instrument, equipped with two 90.05 mL rotors (180.1 mL total column volume). The maximal rotation speed of the rotors was 3000 1/min (280 g), the maximum pressure was 100 bar. Both columns consisted of 10 stainless-steel discs in parallel arrangement, with 90 twin-cells on each disk. The transfer of the stationary and mobile phases and the injection of the sample was carried out using four Gilson gradient HPLC pump (50 mL/min maximal flow rate), controlled by the Armen Elite Glider software. The effluents were monitored using two ECOM DAD600 2WL 200-600 nm UV detector, the product fractions were collected using two Armen LS 5600 fraction collectors.

The solvent system utilized was the n-heptane/2-methyltetrahydrofuran/N,N-dimethyl-formamide/water 3/6/1.3/4 (v/v/v/v) selected based on the partition coefficient measurement (for the partition coefficients see Table 1, solvent system #20). Prior to the CPC run the solvent system was mixed in a separatory funnel. After equilibrium was established, phases were separated.

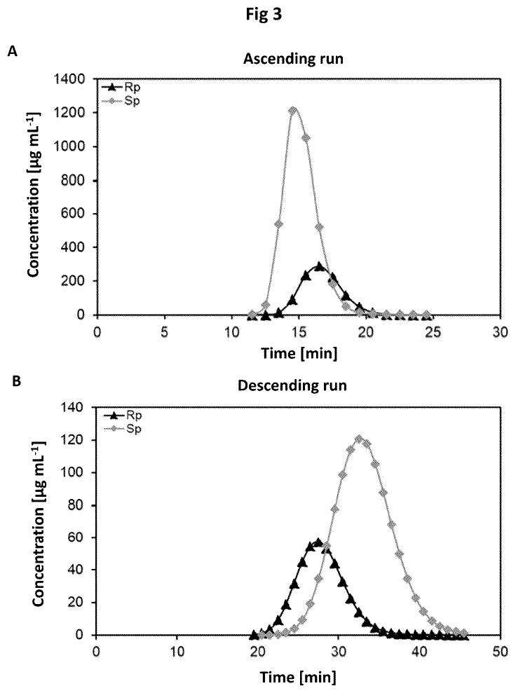

After the determination of the applicable maximum flow rate (10 mL/min) and rotor speed (1700 1/min) that can be used in the MDM mode, ensuring 50% stationary phase retention, further optimal running parameters were determined by an ascending (FIG. 3/A) and descending (FIG. 3/B) run performed in the classic elution mode (we injected 5 mL of remdesivir isomeric mixture dissolved in a 5 mg/mL concentration in a 1/1 (v/v) mixture of the lower and upper phases). The compositions of the fractions of the descending run were determined using the HPLC/DAD/MS technique and by combining fractions #17-27, a 98.4% pure S_{P} isomer was isolated with a yield of 36.6% (see Table 3).

Afterwards, using the data of the two runs in classical elution in the equilibrium cell model simulations, and carried out the simulations of the trapping MDM runs while systematically changing the operating parameters (modelling a three-component mixture: component A: impurity with a shorter retention time than the R_{P} isomer, component B: the R_{P} isomer, component C: the S_{P} isomer) to find the optimal settings for obtaining R_{P} remdesivir with a purity of at least 95%. Using the hereby obtained optimal parameters (Table 4) we then performed the trapping MDM run.

TABLE 4
Results of the simulation of the trapping MDM run.

| | | |
|---|---|---|
| Parameter | Symbol | value |

Loading cycle

| | | |
|---|---|---|
| position of band | x_{fill-up} | 0.25 |
| time of fill-up, dsc. mode | t_{fill-up, dsc} | 3.45 min |
| time of fill-up, asc. mode | t_{fill-up, asc} | 2.07 min |

Separation stage

| | | |
|---|---|---|
| position of band | xₛₑₚₐᵣₐₜᵢₒₙ | 0.40 |
| time of separation step, dsc. mode, | t_{separation, dsc}¹ | 2.07 min |
| 1^{st} cycle | | |
| time of separation step, dsc. mode, | t_{separation, dsc}^{2→N} | 4.14 min |
| 2^{nd} →N^{th} cycles | | |
| time of separation step, asc. mode, | t_{separation, asc}^{2→N} | 2.48 min |
| 2^{nd} →N^{th} cycles | | |
| number of cycles | Nc | 20 |

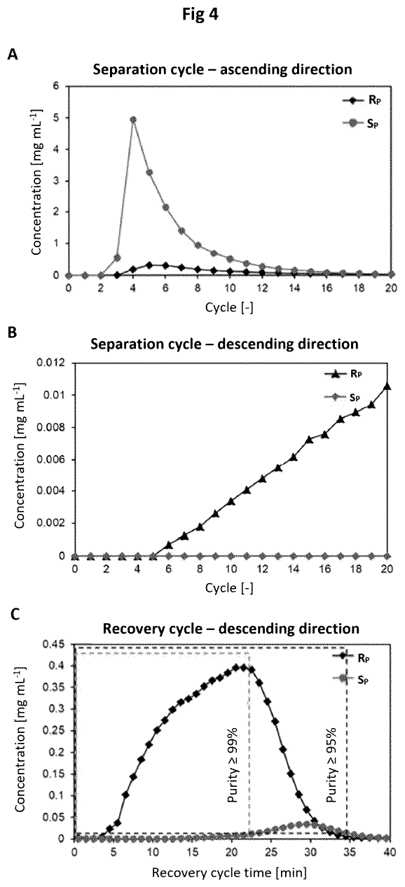

For the trapping MDM run, the isomeric mixture of remdesivir was dissolved at a concentration of 10 mg/mL in the 1/1 (v/v) mixture of the upper and lower phases. Afterwards, we first filled the columns with a 1/1 (v/v) mixture of the lower and upper phases, then in the 0^{th} cycle, first in descending and then ascending mode, the sample was injected for the time indicated in Table 4 using the phase corresponding to the mobile phase. We then ran 20 consecutive descending-ascending cycles with the lengths shown in Table 4, followed by the recovery step. The offline reconstructed chromatograms of the run are included in FIG. 4, the compositions of the fractions were determined using HPLC/DAD/MS technique. From the fractions collected in the recovery step, depending on the desired purity, a) by combining fractions #1-34, 67 mg of 95.1% pure R_{P} isomer could be recovered with a yield of 54.5%, or b) by combining fractions #1-22, it was possible to recover 47 mg of 99.0% pure R_{P} isomer with a yield of 38.6%. The HPLC chromatogram and ³¹P-NMR spectrum of the product obtained in case b) are shown in FIGS. 1/C and 2/C

### Example 2

The aim of the experiment was the separation of phosphorus stereoisomers of remdesivir to obtain high-purity S_{P} isomer in MDM mode, on a laboratory-scale dual-rotor CPC device. The remdesivir isomer mixture to be separated was obtained commercially from Omgene Life Sciences. Based on HPLC/DAD/MS measurements it consisted of 24.14% R_{P} isomer, 75.70% S_{P} isomer and 0.16% other impurity (HPLC chromatogram: FIG. 1/A, ³¹P-NMR spectra: FIG. 2/A). The solvent system utilized was the n-hexane/ethyl acetate/100 mM SBECD, pH 3.0 citrate buffer 1/4/5 (v/v/v) (for the partition coefficients see Table 1, solvent system #4). Prior to the CPC run the solvent system was mixed in a separatory funnel. After equilibrium was established, phases were separated. The sample to be separated was dissolved in the upper phase of the applied solvent system, obtaining a stock solution at 15 mg/mL concentration.

The experiment was carried out on a Gilson PLC 2250 instrument, equipped with two Gilson CPC-250 rotors (500 mL total column volume), controlled by the Gilson Glider CPC software. The columns were filled with a 1/1 (v/v) mixture of the lower and upper phases, then the sample was injected into one of the rotors using the built-in 10-mL loop of the instrument. Afterwards, the CPC was operated in the so-called multiple dual mode (MDM), starting in descending direction we ran one and a half cycles (dsc-asc-dsc). After the determination of the applicable maximum flow rate and rotor speed that can be used in the MDM mode ensuring 50% stationary phase retention, the optimal switching times were determined experimentally, aiming for the highest possible purity of the target component (S_{P} isomer) as a rule of thumb.

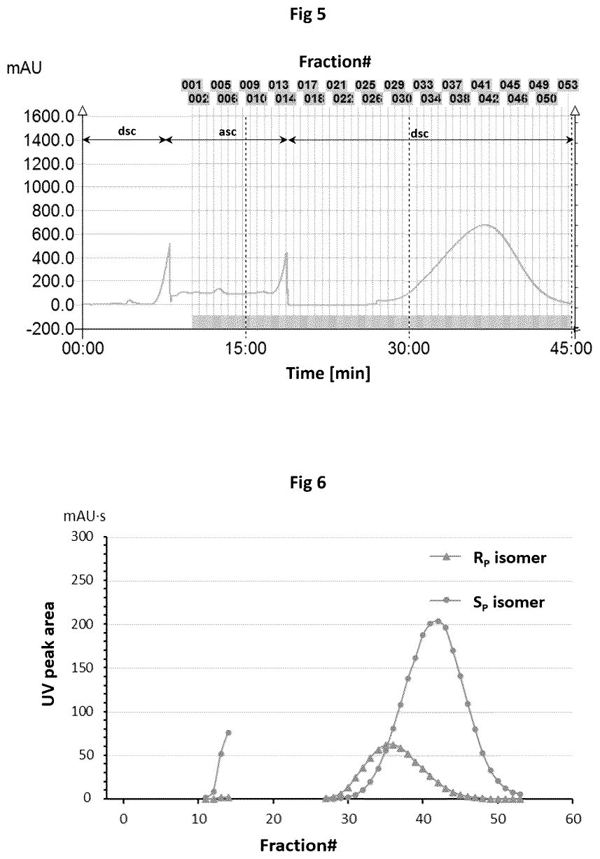

The timetable and main parameters of the CPC method are summarized in Table 5. The CPC chromatogram of the run is presented in FIG. 5, while the corresponding elution profile (fractogram) of the run is provided in FIG. 6.

TABLE 5
Timetable and main parameters of the CPC run for the separation
of the isomeric mixture containing 24.14% R_{P} isomer and 75.70% S_{P}
isomer of remdesivir on a laboratory-scale CPC in MDM mode.

| | | Pump, | Pump, | | | |
|---|---|---|---|---|---|---|
| | Volumetric | Channel A | Channel B | Rotation | Detector | |
| Time | flow | (upper | (lower | speed | wavelength | |
| (min) | (mL/min) | phase) % | phase) % | (1/min) | (nm) | Mode |
| 0.0-8.0 | 30 | 0 | 100 | 1800 | 254 | dsc |
| 8.0-18.6 | 30 | 100 | 0 | 1800 | 254 | asc |
| 18.6-46.0 | 30 | 0 | 100 | 1800 | 254 | dsc |

After the CPC run, the collected fractions were analyzed using HPLC/DAD/MS to determine their R_{P} and S_{P} isomer content. Fractions containing mostly the S_{P} isomer were pooled and evaporated to dryness on a rotary evaporator (laboratory-scale Heidolph Hei-VAP Value with Vacuubrand 4c vacuum pump controlled by Vacuubrand CVC 3000 vacuum control unit). The obtained final product was redissolved in LC-MS grade methanol and analyzed by HPLC/DAD/MS to determine its purity, based on which it was a 95.7% pure S_{P} isomer. During the run, a yield of 53.8% and a productivity of 170.2 mg/L_{column}/h could be achieved relative to the S_{P} isomer.

### Example 3

The aim of the experiment was the separation of phosphorus stereoisomers of remdesivir to obtain high purity S_{P} and R_{P} isomers in MDM mode, on a laboratory-scale dual-rotor CPC device. The remdesivir isomer mixture to be separated was obtained commercially from Omgene Life Sciences. Based on HPLC/DAD/MS measurements it consisted of 43.18% R_{P} isomer, 56.12% S_{P} isomer and 0.7% other impurity (HPLC chromatogram: FIG. 1/B, ³¹P-NMR spectra: FIG. 2/B). The solvent system utilized was the ethyl acetate/100 mM SBECD, pH 2.5 citrate buffer 1/1 (v/v) (for the partition coefficients see Table 1, solvent system #1). Prior to the CPC run the solvent system was mixed in a separatory funnel. After equilibrium was established, phases were separated. The sample to be separated was dissolved in the upper phase of the applied solvent system, obtaining a stock solution at 15 mg/mL concentration.

The experiment was carried out on a Gilson PLC 2250 instrument, equipped with two Gilson CPC-250 rotors (500 ml total column volume), controlled by the Gilson Glider CPC software. The columns were filled with a 1/1 (v/v) mixture of the lower and upper phases, then the sample was injected to one of the rotors using the built-in 10-mL loop of the instrument. Afterwards, the CPC was operated in the so-called multiple dual mode (MDM), starting in descending direction we ran two and a half cycles (dsc-asc-dsc-asc-dsc), after which the sample fraction still retained in the CPC system was extruded in descending mode by pumping the stationary phase through the column. After the determination of the applicable maximum flow rate and rotor speed that can be used in the MDM mode ensuring 50% stationary phase retention, the optimal switching times were determined experimentally, as a rule of thumb aiming for the highest possible yield of the rare target component (R_{P} isomer) in descending mode, while aiming for the highest possible purity of the target component in ascending mode.

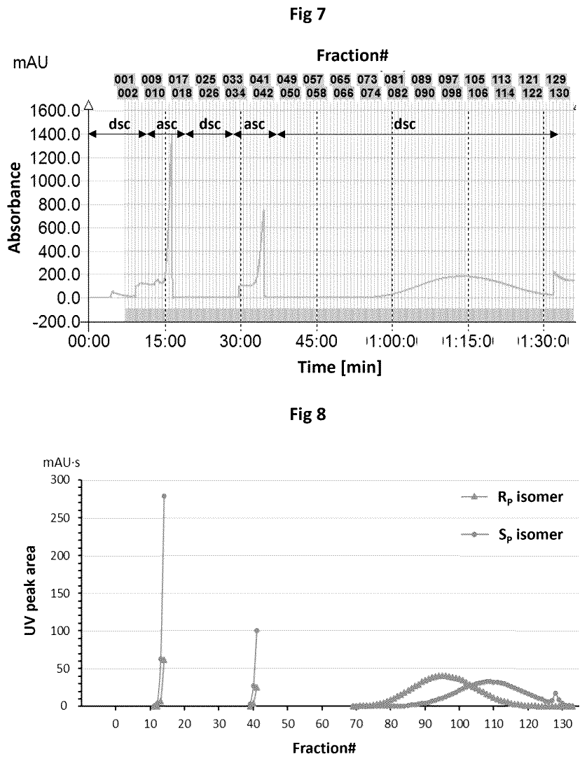

The timetable and main parameters of the CPC method are summarized in Table 6. The CPC chromatogram of the run is presented in FIG. 7, while the corresponding elution profile (fractogram) of the run is provided in FIG. 8.

TABLE 6
Timetable and main parameters of the CPC run for the separation
of the isomeric mixture containing 43.18% R_{P} isomer and 56.12% S_{P}
isomer of remdesivir on a laboratory-scale CPC in MDM mode.

| | | Pump, | Pump, | | | |
|---|---|---|---|---|---|---|
| | Volumetric | Channel A | Channel B | Rotation | Detector | |
| Time | flow | (upper | (lower | speed | wavelength | |
| (min) | (mL/min) | phase) % | phase) % | (1/min) | (nm) | mode |
| 0.0-9.0 | 30 | 0 | 100 | 1800 | 254 | dsc |
| 9.0-16.3 | 30 | 100 | 0 | 1800 | 254 | asc |
| 16.3-27.2 | 30 | 0 | 100 | 1800 | 254 | dsc |
| 27.2-34.5 | 30 | 100 | 0 | 1800 | 254 | asc |
| 34.5-86.0 | 30 | 0 | 100 | 1800 | 254 | dsc |
| 86.0-96.0 | 30 | 100 | 0 | 1800 | 254 | dsc |
| | | | | | | (extrusion) |

After the CPC run, the collected fractions were analyzed using HPLC/DAD/MS to determine their R_{P} and S_{P} isomer content. Fractions containing mostly the R_{P} and S_{P} isomer (>85% R_{P} and S_{P} isomer content) were pooled and evaporated to dryness on a rotary evaporator (laboratory-scale Heidolph Hei-VAP Value with Vacuubrand 4c vacuum pump controlled by Vacuubrand CVC 3000 vacuum control unit). The obtained final products were redissolved in LC-MS grade methanol and analyzed by HPLC/DAD/MS to determine their purity, based on which they were a 95.8% pure R_{P} isomer and a 93.1% pure S_{P} isomer. During the run, a yield of 19.2% and a productivity of 14.7 mg/L_{column}/h could be achieved relative to the R_{P} isomer, while a yield of 18.5% and a productivity of 19.0 mg/L_{column}/h could be achieved relative to the S_{P} isomer.

### Example 4

The aim of the experiment was the separation of phosphorus stereoisomers of remdesivir to obtain high purity S_{P} and R_{P} isomers in MDM mode, on a laboratory-scale CPC device. The remdesivir isomer mixture to be separated was obtained commercially from Omgene Life Sciences. Based on HPLC/DAD/MS measurements it consisted of 24.14% R_{P} isomer, 75.70% S_{P} isomer and 0.16% other impurity (HPLC chromatogram: FIG. 1/A, ³¹P-NMR spectra: FIG. 2/A). The solvent system utilized was the n-heptane/2-methyltetrahydrofuran/N,N-dimethylformamide/water 3/6/1.3/4 (v/v/v/V) (for the partition coefficients see Table 1, solvent system #20). Prior to the CPC run the solvent system was mixed in a separatory funnel. After equilibrium was established, phases were separated. The sample to be separated was dissolved in the upper phase of the applied solvent system, obtaining a stock solution at 25 mg/mL concentration.

The experiment was carried out on a Gilson PLC 2250 instrument, equipped with two Gilson CPC-250 rotors (500 mL total column volume), controlled by the Gilson Glider CPC software. The columns were filled with a 1/1 (v/v) mixture of the lower and upper phases, then the sample was injected into one of the rotors using the built-in 10-mL loop of the instrument. Afterwards, the CPC was operated in the so-called multiple dual mode (MDM), starting in descending direction we ran three cycles (dsc-asc-dsc-asc-dsc). After the determination of the applicable maximum flow rate and rotor speed that can be used in the MDM mode ensuring 50% stationary phase retention, the optimal switching times were determined experimentally, as a rule of thumb aiming for the highest possible yield of the rare target component (R_{P} isomer) in descending mode, while aiming for the highest possible purity of the target component in ascending mode.

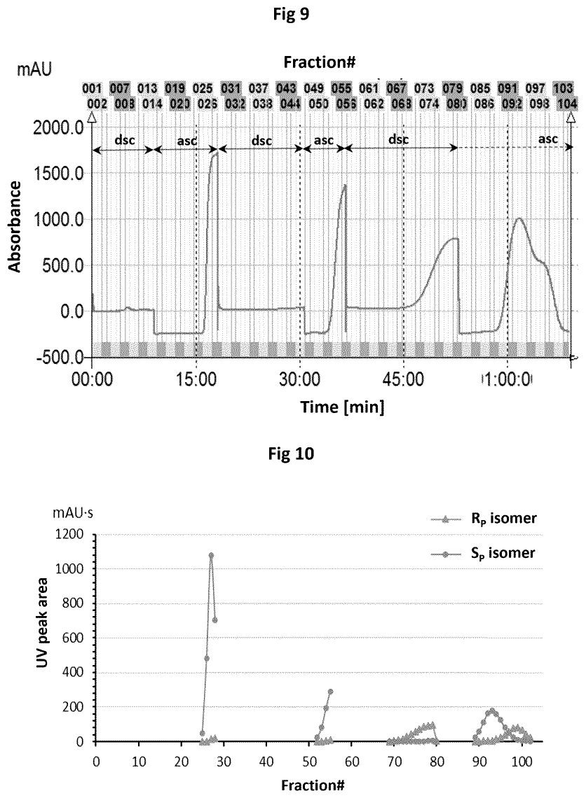

The timetable and main parameters of the CPC method are summarized in Table 7. The CPC chromatogram of the run is presented in FIG. 9, while the corresponding elution profile (fractogram) of the run is provided in FIG. 10.

TABLE 7
Timetable and main parameters of the CPC run for the separation
of the isomeric mixture containing 24.14% R_{P} isomer and 75.70% S_{P}
isomer of remdesivir on a laboratory-scale CPC in MDM mode.

| | | Pump, | Pump, | | | |
|---|---|---|---|---|---|---|
| | | Channel | Channel | | | |
| | | A | B | | | |
| | Volumetric | (upper | (lower | Rotation | Detector | |
| Time | flow | phase) | phase) | speed | wavelength | |
| (min) | (mL/min) | % | % | (1/min) | (nm) | mode |
| 0.0-8.5 | 30 | 0 | 100 | 2000 | 254 | dsc |
| 8.5-18.0 | 30 | 100 | 0 | 2000 | 254 | asc |
| 18.0-30.3 | 30 | 0 | 100 | 2000 | 254 | dsc |
| 30.3-36.3 | 30 | 100 | 0 | 2000 | 254 | asc |
| 36.3-53.0 | 30 | 0 | 100 | 2000 | 254 | dsc |
| 53.0-90.0 | 30 | 100 | 0 | 2000 | 254 | asc |

After the CPC run, the collected fractions were analyzed using HPLC/DAD/MS to determine their R_{P} and S_{P} isomer content. Fractions containing mostly the R_{P} and S_{P} isomer (>85% R_{P} and S_{P} isomer content) were pooled and evaporated to dryness on a rotary evaporator (laboratory-scale Heidolph Hei-VAP Value with Vacuubrand 4c vacuum pump controlled by Vacuubrand CVC 3000 vacuum control unit). The obtained final products were redissolved in LC-MS grade methanol and analyzed by HPLC/DAD/MS to determine their purity, based on which they were a 95.5% pure R_{P} isomer and a 97.8% pure S_{P} isomer. During the run, a yield of 72.3% and a productivity of 78.3 mg/L_{column}/h could be achieved relative to the R_{P} isomer, while a yield of 92.0% and a productivity of 305.4 mg/L_{column}/h could be achieved relative to the S_{P} isomer.

### Example 5

The aim of the experiment was the separation of phosphorus stereoisomers of remdesivir to obtain high purity S_{P} and R_{P} isomers in MDM mode, on a laboratory-scale dual-rotor CPC device. The remdesivir isomer mixture to be separated was obtained commercially from Omgene Life Sciences. Based on HPLC/DAD/MS measurements it consisted of 43.18% R_{P} isomer, 56.12% S_{P} isomer and 0.7% other impurity (HPLC chromatogram: FIG. 1/B, ³¹P-NMR spectra: FIG. 2/B). The solvent system utilized was the n-heptane/2-methyltetrahydrofuran/N,N-dimethylformamide/water 3/6/1.3/4 (v/v/v/v) (for the partition coefficients see Table 1, solvent system #20). Prior to the CPC run the solvent system was mixed in a separatory funnel. After equilibrium was established, phases were separated. The sample to be separated was dissolved in the upper phase of the applied solvent system, obtaining a stock solution at 25 mg/ml concentration.

The experiment was carried out on a Gilson PLC 2250 instrument, equipped with two Gilson CPC-250 rotors (500 mL total column volume), controlled by the Gilson Glider CPC software. The columns were filled with a 1/1 (v/v) mixture of the lower and upper phases, then the sample was injected into one of the rotors using the built-in 10-mL loop of the instrument. Afterwards, the CPC was operated in the so-called multiple dual mode (MDM), starting in descending direction we ran one and a half cycles (dsc-asc-dsc). After the determination of the applicable maximum flow rate and rotor speed that can be used in the MDM mode ensuring 50% stationary phase retention, the optimal switching times were determined experimentally, as a rule of thumb aiming for the highest possible yield of the rare target component (R_{P} isomer) in descending mode, while aiming for the highest possible purity of the target component in ascending mode.

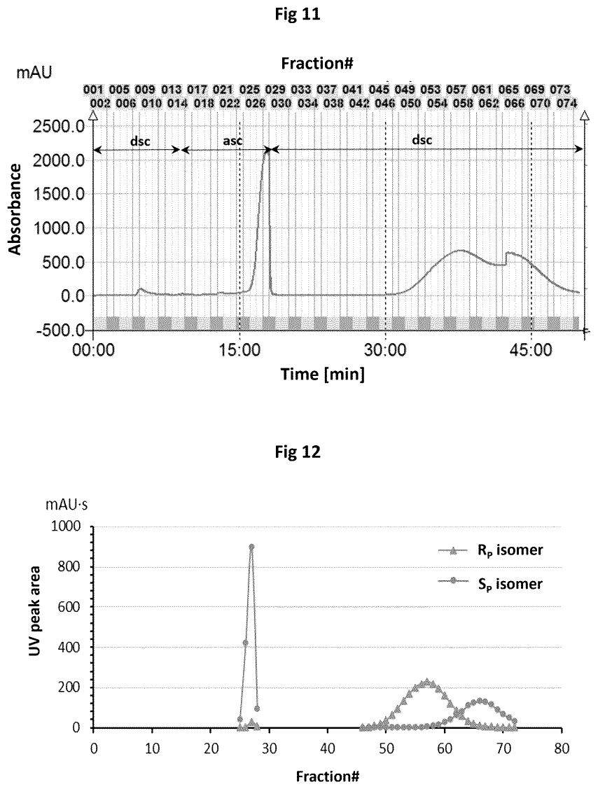

The timetable and main parameters of the CPC method are summarized in Table 8. The CPC chromatogram of the run is presented in FIG. 11, while the corresponding elution profile (fractogram) of the run is provided in FIG. 12.

TABLE 8
Timetable and main parameters of the CPC run for the separation
of the isomeric mixture containing 43.18% R_{P} isomer and 56.12% S_{P}
isomer of remdesivir on a laboratory-scale CPC in MDM mode.

| | | Pump, | Pump, | | | |
|---|---|---|---|---|---|---|
| | Volumetric | Channel A | Channel B | Rotation | Detector | |
| Time | flow | (upper | (lower | speed | wavelength | |
| (min) | (mL/min) | phase) % | phase) % | (1/min) | (nm) | mode |
| 0.0-8.5 | 30 | 0 | 100 | 2000 | 254 | dsc |
| 8.5-18.0 | 30 | 100 | 0 | 2000 | 254 | asc |
| 18.0-50.0 | 30 | 0 | 100 | 2000 | 254 | dsc |

After the CPC run, the collected fractions were analyzed using HPLC/DAD/MS to determine their R_{P} and S_{P} isomer content. Fractions containing mostly the R_{P} and S_{P} isomer (>85% R_{P} and S_{P} isomer content) were pooled and evaporated to dryness on a rotary evaporator (laboratory-scale Heidolph Hei-VAP Value with Vacuubrand 4c vacuum pump controlled by Vacuubrand CVC 3000 vacuum control unit). The obtained final products were redissolved in LC-MS grade methanol and analyzed by HPLC/DAD/MS to determine their purity, based on which they were a 95.6% pure R_{P} isomer and a 95.7% pure S_{P} isomer. During the run, a yield of 81.5% and a productivity of 220.7 mg/L_{column}/h could be achieved relative to the R_{P} isomer, while a yield of 85.3% and a productivity of 300.2 mg/L_{column}/h could be achieved relative to the S_{P} isomer.

### Example 6

The aim of the experiment was the separation of phosphorus stereoisomers of remdesivir to obtain high purity S_{P} and R_{P} isomers in MDM mode, on a pilot-scale dual-rotor CPC device. The remdesivir isomer mixture to be separated was obtained commercially from Omgene Life Sciences. Based on HPLC/DAD/MS measurements it consisted of 43.18% R_{P} isomer, 56.12% S_{P} isomer and 0.7% other impurity (HPLC chromatogram: FIG. 1/B, ³¹P-NMR spectra: FIG. 2/B). The solvent system utilized was the n-heptane/2-methyltetrahydrofuran/N,N-dimethylformamide/water 3/6/1.3/4 (v/v/v/v) (for the partition coefficients see Table 1, solvent system #20). Prior to the CPC run the solvent system was mixed in a separatory funnel. After equilibrium was established, phases were separated. The sample to be separated was dissolved in the upper phase of the applied solvent system, obtaining a stock solution at 25 mg/ml concentration.

The experiment was carried out on RotaChrom pilot-scale dual-rotor CPC (rCPC) system (ECOM ECB2005PC Gradient Box with PC, ECOM ECP 2300 Isocratic Pump, ECOM Flash 14 DAD 400 UV-detector, ECOM Box with 10-position valve fraction collector and two RotaChrom rCPC rotor (4.2 L total internal volume, 200 extraction cells)) controlled by the ECOMAC Software. The columns were filled with a 1/1 (v/v) mixture of the lower and upper phases, then equilibrated in descending mode. This was followed by the injection of the sample using channel C of the pump. After injection, the CPC was operated in the so-called multiple dual mode (MDM), starting in descending direction we ran one and a half cycles (dsc-asc-dsc). The optimal switching times were determined experimentally, as a rule of thumb aiming for the highest possible yield of the rare target component (R_{P} isomer) in descending mode, while aiming for the highest possible purity of the target component in ascending mode.

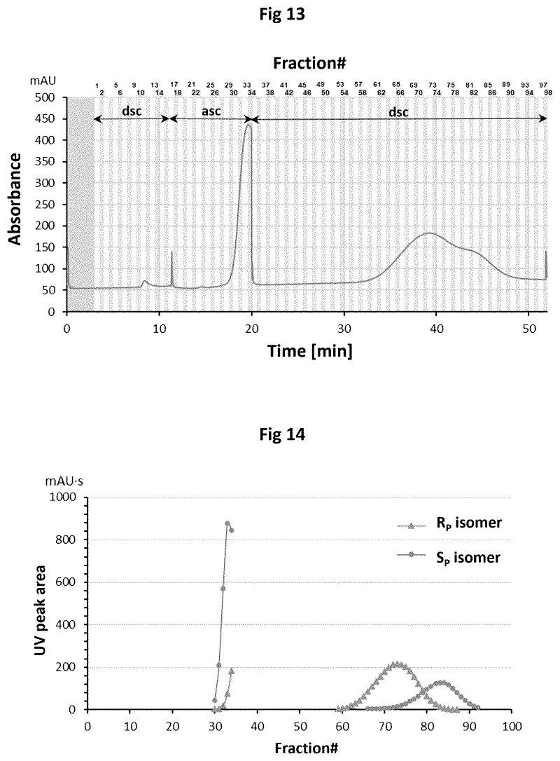

The timetable and main parameters of the CPC method are summarized in Table 9. The CPC chromatogram of the run is presented in FIG. 13, while the corresponding elution profile (fractogram) of the run is provided in FIG. 14.

TABLE 9
Timetable and main parameters of the CPC run for the separation of the isomeric mixture containing
43.18% R_{P} isomer and 56.12% S_{P} isomer of remdesivir on a pilot-scale CPC in MDM mode.

| | | Pump, | Pump, | | | | |
|---|---|---|---|---|---|---|---|
| | | Channel | Channel | Pump, | | | |
| | Volumetric | A | B | Channel | Rotation | Detector | |
| Time | flow | (upper | (lower | C | speed | wavelength | |
| (min) | (mL/min) | phase) % | phase) % | (sample) % | (1/min) | (nm) | mode |
| 0.0-2.5 | 250 | 0 | 100 | 0 | 1000 | 254 | equilibration |
| 2.5-3.0 | 160 | 0 | 0 | 100 | 1000 | 254 | injection |
| | | | | | | | of sample |
| 3.0-11.2 | 250 | 100 | 0 | 0 | 1000 | 254 | dsc |
| 11.2-20.0 | 250 | 0 | 100 | 0 | 1000 | 254 | asc |
| 20.0-52.0 | 250 | 100 | 0 | 0 | 1000 | 254 | dsc |

After the CPC run, the collected fractions were analyzed using HPLC/DAD/MS to determine their R_{P} and S_{P} isomer content. Fractions containing mostly the R_{P} and S_{P} isomer (>85% R_{P} and S_{P} isomer content) were pooled and evaporated to dryness on a rotary evaporator (laboratory-scale Heidolph Hei-VAP Value with Vacuubrand 4c vacuum pump controlled by Vacuubrand CVC 3000 vacuum control unit). The obtained final products were redissolved in LC-MS grade methanol and analyzed by HPLC/DAD/MS to determine their purity, based on which they were a 95.0% pure R_{P} isomer and a 95.1% pure S_{P} isomer. During the run, a yield of 65.4% and a productivity of 169.2 mg/L_{column}/h could be achieved relative to the R_{P} isomer, while a yield of 58.3% and a productivity of 195.7 mg/L_{column}/h could be achieved relative to the S_{P} isomer.

1. A method for the separation of phosphorus stereoisomers of remdesivir, wherein R_{P} and/or S_{P} remdesivir is/are obtained from an R_{P}-S_{P} remdesivir isomeric mixture by liquid-liquid chromatography (LLC) using a biphasic, binary, ternary or quaternary solvent system, wherein
the binary solvent system consists of the following phase-forming components:
an alkanoate ester with 3-6 carbon atoms; and
an aqueous solution, containing a solubility-enhancing additive, and a buffer salt and/or an organic acid used for setting the pH of the aqueous phase in the range of pH 2-7;
the ternary solvent system consists of the following phase-forming components:
an aliphatic alkane with 5-8 carbon atoms;
a furane-type aromatic heterocyclic compound or an alkanoate ester with 3-6 carbon atoms;
an aqueous solution, containing a solubility-enhancing additive and/or a buffer salt and/or an organic acid used for setting the pH of the aqueous phase in the range of pH 2-7;
the quaternary solvent system consists of the following phase-forming components:
an aliphatic alkane with 5-8 carbons;
a furane-type aromatic heterocyclic compound;
a further organic co-solvent selected from an aliphatic alcohol with 2-5 carbons, preferably methanol or ethanol; a ketone with 2-4 carbons, preferably acetone; an organic nitrile with 2-3 carbons, preferably acetonitrile; dimethyl sulfoxide; N,N-dimethylformamide; N,N-dimethylacetamide; and 1,2-dimethoxyethane;
water, or an aqueous solution containing a solubility-enhancing additive and/or a buffer salt and/or an organic acid used for setting the pH of the aqueous phase in the range of pH 2-7.

2. The method of claim 1, wherein the alkanoate ester is ethyl acetate, n-propyl acetate, isopropyl acetate or n-butyl acetate.

3. The method of claim 1, wherein the furane-type aromatic heterocyclic compound is 2-methyltetrahydrofuran.

4. The method of claim 1, wherein the solubility-enhancing additive is a cyclodextrin or its derivative, preferably sulfobutylether-ß-cyclodextrin or a polyethylene glycol, preferably polyethylene glycol-1500.

5. The method of claim 1, wherein the buffer salt is citrate or phosphate buffer salt; and the organic acid is formic acid, acetic acid or trifluoroacetic acid.

6. The method of claim 1, wherein the LLC separation is realized in a hydrodynamic counter-current chromatography (CCC) device or in a hydrostatic centrifugal partition chromatography (CPC) device, preferably in a dual-rotor CPC device.

7. The method of claim 1, wherein the solvent system is selected from the group consisting of ethyl acetate/100 mM sulfobutylether-ß-cyclodextrin, pH 2.0 phosphate buffer in a 1/1 (v/v) ratio; n-hexane/ethyl acetate/100 mM sulfobutylether-ß-cyclodextrin, pH 3.0 citrate buffer in a 1/1/1 (v/v/v) ratio; and n-heptane/2-methyltetrahydrofuran/N,N-dimethylformamide/water in a 3/6/1.3/4 (v/v/v/v) ratio.

8. The method of claim 1, wherein the LLC separation is realized in classical elution mode using descending or ascending mode.

9. The method of claim 1, wherein the LLC separation is realized in trapping multiple dual mode (MDM), where the isomer mixture is dissolved in both phases of the solvent system and injected between the two LLC columns according to the elution mode: during ascending run the upper phase, while during descending run the lower phase is injected.

10. The method of claim 1, wherein the LLC separation is realized in multiple dual mode (MDM), where the isomer mixture is dissolved in either the upper or the lower phase of the solvent system and injected between the two columns of the LLC instrument followed by elution in MDM mode.

11. The method of claim 1, wherein by collecting and combining adequate product fractions>95% pure R_{P} and/or S_{P} isomer(s) is/are obtained.
